SEQUENCE LISTING (13 sequences)

SEQ ID NO: 1 - DNA, 623 bp, Homo sapiens
ctattgtcaagtcacaccttccctgcctgtgactggcacagacattagccaatgggtgcttggatagggtgcgccggcgccccgccccccttagcgccccgccgccccgaggcaccctctggcagactcggcggcggcgacagcctgggcggacagtgcgccgtgcgcaggcgcggagctagacctcgctgcagcccccatcgcctcggggagtctcacccaccgagtccgcccgctggcccgtcagtgctctccccgtcgtttgccctctccagttcccccagtgcctgccctacgcaccccgatggcggagctgcggcctagcggcgcccccggccccaccgcgcccccggcccctggcccgactgcccccccggccttcgcttcgctctttcccccgggactgcacgccatctacggagagtgccgccgcctttaccctgaccagccgaacccgctccaggttaccgctatcgtcaagtactggtatgctctgggccgcggggagacggacaggcgcgggctggaaagggttaaagcgccgagggcgaagtaatttgtgggaggtgggaggaggggtagagaatggttaaagcactcagaaggagggcttcttgctgcga

SEQ ID NO: 2 - DNA, 264 bp, Homo sapiens
caaagtagagcgccttagcttgacattgtctgatttccaggcttacactaacacccctgtgttttgttttttgcaggttgggtggcccagaccccttggactatgttagcatgtacaggaatgtggggagcccttctgctaacatccccgagcactggcactacatcagcttcggcctgagtgatctctatggtgacaacagagtccatgagtgagtatatgccacctgttctttatccagagccttattcctgaggtcttcct

SEQ ID NO: 3 - DNA, 275 bp, Homo sapiens
gggagaactttagactttcaagagagtgtttttcctaaggtaattgagcttaaaacacttgctttttatgtctttcaggtttacaggaacagatggacctagtggttttggctttgagttgacctttcgtctgaagagagaaactggggagtctgccccaccaacatggcccgcagagttaatgcagggcttggcacgatacgtgttccagtcaggtaggaggccagggctggctgctgtgctggtccttttgccatgagcctggttgactttga

SEQ ID NO: 4 - DNA, 400 bp, Homo sapiens
aagcctcccagcctgggctagtgagatcccagcccagattccaggcctggatctggggccttgaacaatgaggatccttgtatctctcccacagagaacaccttctgcagtggggaccatgtgtcctggcacagccctttggataacagtgagtcaagaattcagcacatgctgctgacagaggacccacagatgcagcccgtgcagacaccctttggggtagttaccttcctccaggtgaggcacaggttggacgctggctcaagccttcctgtgggaagggtcctgggaggacaaggaggcttgaggagggggagtgaagggagtgggaggtttcctttggcctcgtcctgatttctgtttcctctgatggtttgtcaggtagattcagatggtctga

SEQ ID NO: 5 - DNA, 205 bp, Homo sapiens
tggggggtggccattaacacacaatgggctttctatcctgggcctcagatcgttggtgtctgcactgaagagctacactcagcccagcagtggaacgggcagggcatcctggagctgctgcggacagtgcctatgtgagtacccatgcaaggtgggagcgcggctccctgggcctgggggtgggagtccctccactaccctccat

SEQ ID NO: 6 - DNA, 286 bp, Homo sapiens
tccctgaccacgaactattcccctgtgtcctaggcctggggcagcaaacagggcaggctgtaggcccagcccatcagccccagaccctcagttaccattgtatcccctttccttgtccacagtgctggcggcccctggctgataactgacatgcggaggggagagaccatatttgagatcgatccacacctgcaagtatgtcttgagtgaggaaaacctttctagcaccctgtgcctaggcctcttccaaataacactggctttcatcctgggaaaacagaggacc

SEQ ID NO: 7 - DNA, 361 bp, Homo sapiens
ttgtcccatgctcagcaccacaagggctcagtaaatactgtaagagcagtggctgaaagggtggtcaccttgggtcaccagttctctgaaagaactctggctctttggttcttttcaagcaggagagagttgacaaaggcatcgagacagatggctccaacctgagtggtgtcagtgccaagtgtgcctgggatgacctgagccggccccccgaggatgacgaggacagccggagcatctgcatcggcacacagccccggcgactctctggcaaaggtgggagccatcactcagcattccaccagccttcctccttccttttccccagggcctggtttccagtctctctaggatgggtctc

SEQ ID NO: 8 - DNA, 274 bp, Homo sapiens
aggtttcaagagcggggtgagaattgctgggagcccactgggccactgggcaacttagtggtgtcgttgcagacacagagcagatccgggagaccctgaggagaggactcgagatcaacagcaaacctgtccttccaccaatcaaccctcagcggcagaatggcctcgcccacgaccgggccccgtaagttccccagtgtccctgggctggaacaagaggacgacttttttctgaagggcctgtccctgtggattgcatgagagagaacaatgc

SEQ ID NO: 9 - DNA, 266 bp, Homo sapiens
tccctgagcttttcaccttgtgccgaaccttttcctgtgcttgcttcacaggagccgcaaagacagcctggaaagtgacagctccacggccatcattccccatgagctgattcgcacgcggcagcttgagagcgtacatctgaaattcaaccaggagtccggagccctcattcctctctgcctaaggtgagcgagacagccctgccacacagtttaccccacagcacccagctcagcctccagggggcacttcagagcctccccag

SEQ ID NO: 10 - DNA, 375 bp, Homo sapiens
gcctgctgtgcttggaactgtttccaagcccagctcctcactgtctccatgttcccatctccaggggcaggctcctgcatggacggcactttacatataaaagtatcacaggtgacatggccatcacgtttgtctccacgggagtggaaggcgcctttgccactgaggagcatccttacgcggctcatggaccctggttacaagtgagaaggccctttttcttctccctccttcctttcatagacttccttgcccacccctcctcttctcccttggcagctcttgatggcaccccttcctggggggctggtcatgaatgcctcatggattcagggcctggggcctgtgtgtaggtatggagtgtggatgctgcta

SEQ ID NO: 11 - DNA, 275 bp, Homo sapiens
cttccctgtgtcccttgaacagatcacagtgagctcatctctcctccatggtcagaagagaggtataacgcttggtggttggcaaaaagatcatacatttaaaaataataataaaagcctgccttgtgccttcacagattctgttgaccgaagagtttgtagagaaaatgttggaggatttagaagatttgacttctccagaggaagtaagcttgtttgacttttcctgacaacaggtcccgtctctgggaccatgtgtgtgcgtgcgtgtgcac

SEQ ID NO: 12 - DNA, 280 bp, Homo sapiens
ggcctgtcctatccctagctccccggggacaggcctgggcaatctctggaaagaccacggtgtattctgctaaccactcacactcctggtctgtgcttgctccctccacagttcaaacttcccaaagagtacagctggcctgaaaagaagctgaaggtctccatcctgcctgacgtggtgttcgacagtccgctacactagcctgggctgggccctgcagtggccagcagggagcccagctgctccccagtgacttccagtgtaacagttgtgtcaacga

SEQ ID NO: 13 - protein, 484 aa, Homo sapiens
MAELRPSGAPGPTAPPAPGPTAPPAFASLFPPGLHAIYGECRRLYPDQPNPLQVTAIVKYWLGGPDPLDYVSMYRNVGSPSANIPEHWHYISFGLSDLYGDNRVHEFTGTDGPSGFGFELTFRLKRETGESAPPTWPAELMQGLARYVFQSENTFCSGDHVSWHSPLDNSESRIQHMLLTEDPQMQPVQTPFGVVTFLQIVGVCTEELHSAQQWNGQGILELLRTVPIAGGPWLITDMRRGETIFEIDPHLQERVDKGIETDGSNLSGVSAKCAWDDLSRPPEDDEDSRSICIGTQPRRLSGKDTEQIRETLRRGLEINSKPVLPPINPQRQNGLAHDRAPSRKDSLESDSSTAIIPHELIRTRQLESVHLKFNQESGALIPLCLRGRLLHGRHFTYKSITGDMAITFVSTGVEGAFATEEHPYAAHGPWLQILLTEEFVEKMLEDLEDLTSPEEFKLPKEYSWPEKKLKVSILPDVVFDSPLH

(19) United States
(12) Patent Application Publication
(10) Pub. No.: US 2005/0158718 A1
(43) Pub. Date: Jul. 21, 2005

(54) Diagnostic and therapeutic uses of SUFU gene

(76) Inventors: Michael D. Taylor, Missisauga (CA); James Rutka, North York (CA); David Hogg, Toronto (CA); Ling Liu, Toronto (CA)

Correspondence Address: MYERS BIGEL SIBLEY & SAJOVEC, PO BOX 37428, RALEIGH, NC, 27627, US

(21) Appl. No.: 10/462,290

(22) Filed: Jun. 16, 2003

(51) Int. Cl. C12Q 1/68

(52) U.S. Cl. 435/6

(57) ABSTRACT

The invention relates to the identification of the SUFU (supressor of fused) gene as a tumor suppressor gene and the identification of mutations of the SUFU gene that are associated with the development of cancer, particularly medulloblastoma. The invention provides methods for determining a diagnosis, prognosis or risk of a tumor pathology in a subject involving a SUFU gene mutation, where the method comprises detecting a SUFU gene mutation in DNA from a subject. The SUFU gene comprises exons 1 through 12 and the mutation is associated with the tumor pathology.

### RELATED APPLICATIONS

This application claims priority under 35 U.S.C. § 119 (e) to U.S. provisional application Ser. No. 60/388,305, filed Jun. 14, 2002, the entire contents of which are incorporated herein by reference.

### FIELD OF THE INVENTION

The invention relates to diagnostic methods and therapeutic methods and compositions for use in cancer. More specifically, the invention relates to the identification of the SUFU (supressor of fused) gene as a tumor suppressor gene and the identification of mutations of the SUFU gene which is associated with the development of cancer, particularly medulloblastoma.

### BACKGROUND OF THE INVENTION

Brain tumors are the second most common cancer in children after leukemia, with the incidence increasing at a rate of 5 to 10 per cent per year. More than 200 Canadian children are diagnosed with brain tumors each year, with approximately 100 new cases at The Hospital for4Sick Children alone. Despite advances in treatment, survival from brain tumors remains lower than for other forms of cancer. Medulloblastoma is the most common malignant pediatric brain tumor and accounts for 20 percent of all pediatric brain tumors and is more common in boys than girls. It is a rapidly growing tumor arising from the external granular cells of the cerebellum.

The sonic hedgehog (SHH) signal transduction pathway directs the embryonic development of many organs and tissues, including the growth and patterning of the cerebellum (1) in vertebrates. Disruptions at various locations in the SHH pathway have been linked to a variety of malignancies. SHH is a powerful mitogen for external granular cells of the cerebellum, which give rise to medulloblastomas. SHH binds with the receptor PTCH. PTCH is a transmembrane protein that together with SMOH, a seven transmembrane protein, forms a receptor complex for SHH. Ligand binding results in derepression of signaling from SMOH and subsequently to activation of the transcription factor GLI. Expression of GLI is elevated in cells treated with SHH.

Mutations of the SHH receptor gene PTCH occur in 50% or more of individuals with nevoid basal cell carcinoma syndrome (NBCCS) who present at birth with an overgrowth syndrome that includes large body size, hypertelorism, frontal bossing and additional malformations such as a flat nasal bridge, bifid ribs, polydactyly and occasionally developmental delay (3-6). Later in life, individuals with NBCCS may develop a spectrum of neoplasms, including basal-cell carcinoma (BCC), medulloblastoma and meningioma. Somatic mutations of the genes PTCH and SMOH occur in a subset of sporadic BCCs and medulloblastomas (7-13) and human GLI was initially identified as a gene amplified in a human glioblastoma (14). These observations are consistent with those in mice as 14% of PTCH^{±} mice develop medulloblastomas (15, 16), and overexpression of human GLI (17), mouse Gli2 (18) or SHH (19) in mouse skin leads to BCC.

Previous studies have identified a gene, the Suppressor-of-Fused (SUFU) gene, which encodes a protein involved in the GLI-1 signaling pathway (Kogerman et al., Nature Cell Biol., 1:312-319, 1999). The SUFU gene product is a negative regulator of GLI-1 signalling and assists in the nuclear cytoplasmic transport of GLI-1. Both SHH and GLI signaling are involved in brain development. Wild type SUFU sequesters GLI. Mutations have now been identified whereby the normal function of SUFU is disrupted such that transcription continues through GLI unabated.

The SUFU gene has not previously been recognized to play a role in malignancy and furthermore, no underlying mechanisms for the development of medulloblastoma have been previously described.

### SUMMARY OF THE INVENTION

In accordance with the present invention novel mutations of the human SUFU gene have now been identified and associated with the development of medulloblastoma. Thus the SUFU gene has now been identified as a tumor suppressor gene involved in medulloblastoma.

According to the present invention is an isolated human SUFU gene comprising 12 exons, said gene having a mutation in one or more of exons 1, 2, 8 or 9, wherein said mutation is indicative of a medulloblastoma phenotype.

According to another aspect of the invention is an isolated human SUFU gene comprising one or more mutations selected from the group consisting of IVS8+1G→A, IVS1-1G→T, 1129delTCCGGAG, IVS1-1A→T and E1 143insA.

According to another aspect of the invention is an isolated human SUFU gene comprising a SUF(212-484) N-terminal deletion.

According to yet another aspect of the invention is a method for determining a diagnosis, prognosis or risk of a tumor pathology in a subject involving a SUFU gene mutation, said method comprising detecting a SUFU gene mutation in DNA from said subject, wherein said SUFU gene comprises exons 1 through 12 and said mutation is associated with said tumor pathology.

According to yet another aspect of the invention is method for determining whether a subject is at risk for development of medulloblastoma, the method comprising the steps of:
(a) obtaining a nucleic acid sample from the subject; and
(b) conducting an assay on the nucleic acid sample to determine the presence or absence of a Suppressor-of-Fused (SUFU) gene mutation associated with medulloblastoma, wherein the presence of a SUFU gene mutation associated with medulloblastoma indicates that the subject is at risk for development of medulloblastoma.

According to another aspect of the invention is method for determining whether a subject displaying a medulloblastoma phenotype, the method comprising the steps of:
(a) obtaining a nucleic acid sample from the subject;
(b) conducting an assay on the nucleic acid sample to determine the presence or absence of a SUFU gene mutation associated with medulloblastoma, wherein the presence of a SUFU gene mutation associated with medulloblastoma indicates that the subject is suffering from medulloblastoma.

According to still another embodiment of the invention is a method for treating a subject bearing a mutated SUFU gene comprising administering to the subject an effective amount of an agent selected from the group consisting of:
(a) a nucleotide sequence encoding a normal SUFU gene;
(b) normal SUFU protein or an effective fragment thereof;
(c) a compound which inhibits SHH signalling; and
(d) an antibody that binds to a mutant SUFU protein.

According to a further aspect of the invention is a method for screening a candidate compound for its potential as a therapeutic for improvement of SUFU function in a subject having a mutated SUFU gene comprising screening the candidate compound for its ability to inhibit SHH signalling, wherein an ability to inhibit SHH signalling indicates that the compound is a potential therapeutic for said subject.

This invention also provides kits for the detection and/or quantification of SUFU gene or gene product. The kits can include a container containing one or more of identified nucleic acids, amplification primers, and antibodies with or without labels, free, or bound to a solid support as described herein. The kits can also include instructions for the use of one or more of these reagents in any of the assays described herein.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### BRIEF DESCRIPTION OF THE DRAWINGS

Embodiments of the present invention will now be described more fully with reference to the accompanying drawings:

FIG. 1A-1D show mutations of the SUFU gene in desmoplastic medulloblastomas. FIG. 1A shows a conserved GT splice-donor site consensus sequence of exon 8 being mutated to AT in the tumor (IVS8+1G→A). No wild type sequence was seen, suggesting LOH. Analysis by RT-PCR indicated splicing of exon 7 to exon 9 with a subsequent frameshift and premature stop codon (data not shown). FIG. 1 B shows a heterozygous mutation in the conserved AG splice-acceptor site of exon 2, which was mutated to AT (IVS1-1G→T) in the germline DNA. The wild type was absent in a sample from the desmoplastic medulloblastoma of the same individual, consistent with LOH. Analysis by RT-PCR showed splicing of exon 1 to exon 4 with a subsequent frameshift and premature stop codon at the 3′ end of exon 1 (data not shown). FIG. 1C shows that sequence analysis of the tumor DNA showed a deletion of seven nucleotides from exon 9 (1129delTCCGGAG) resulting in a frameshift and premature stop codon, the wild type allele was absent. FIG. 1D shows hematoxylin and eosin staining that identifies histopathologic features of the tumor with the 1129delTCCGGAG mutation. This small blue cell tumor with nodular, reticulin-free zones was typical of a desmoplastic medulloblastoma.

FIG. 2 shows a diagram of the deletion on chromosome 10q in a subject (a child). Multiple FISH experiments using contiguous BAC clones showed that the deletion was approximately 2.5-2.8 Mb in size and included at least 28 genes (not all shown here). FISH using a BAC probe that encompassed BTRC indicated that this gene was not included in the deletion (most centromeric gene in this figure).

FIGS. 3A to 3C show western blots. FIG. 3A shows western blotting of 293 cells, transfected with Flag-tagged GLI on its own or with Myc-tagged wild type SUFU (SUFU-wt), SUFU-Δex8 or SUFU(212-484).

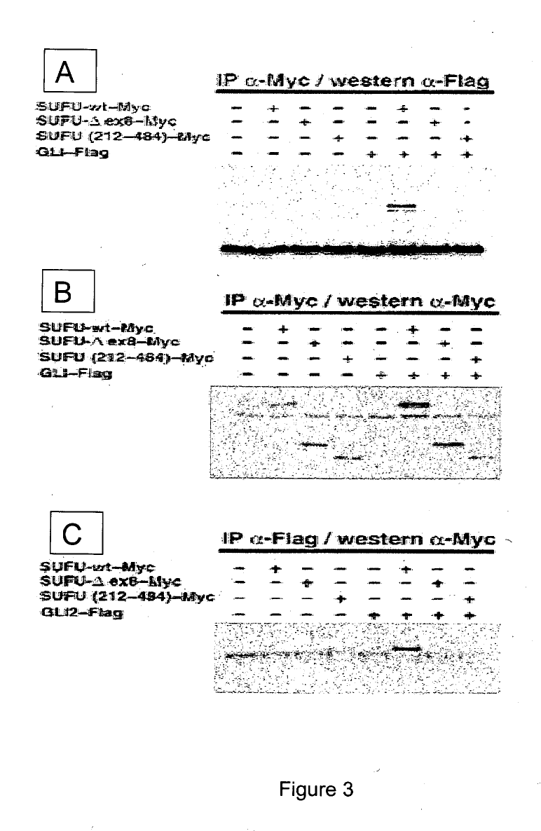

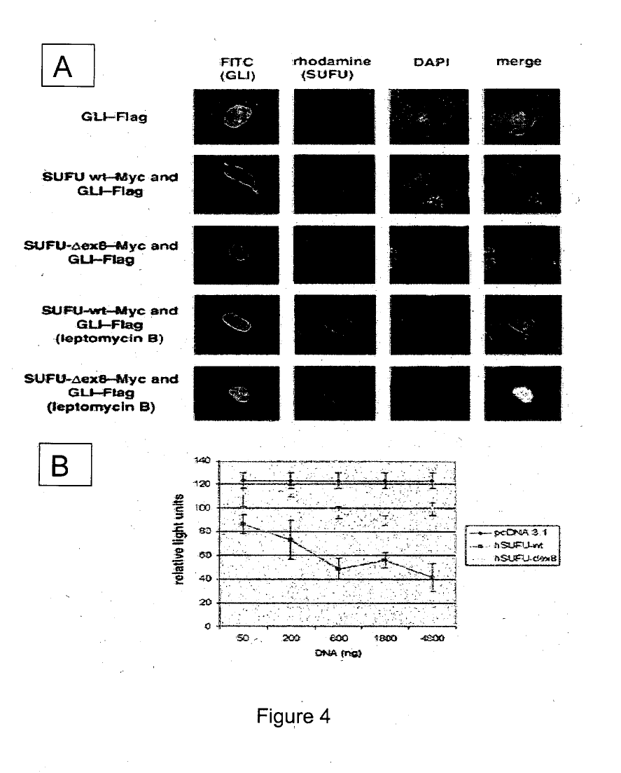

Immunoprecipitation using antibody against the Myc epitope (IP α-Myc), followed by SDS-PAGE and western blotting with antibody against the Flag epitope (western α-Flag), showed that whereas wild type SUFU (SUFU-wt) could bind GLI, the Δex8 mutant and the SUFU(212-484) mutant could not. FIG. 3B shows a Western blot demonstrating the presence of equal amounts of immunoprecipitated wild type SUFU, Δex8 and SUFU(212-484), confirmed by stripping and reprobing the IP α-Myc, western α-Flag blot from FIG. 3A with an antibody against Myc. FIG. 3C shows a Western blot of transfected 293 cells, co-transfected alone and in combination with Flag-tagged GLI2 and Myc-tagged wild type SUFU, SUFU-Δex8 and SUFU(212-484). Immunoprecipitations against the Flag epitope followed by SDS-PAGE and western blotting against the Myc epitope showed that whereas GLI2 immunoprecipitated SUFU-wt, it did not bind SUFU-Δex8 or SUFU(212-484).

FIGS. 4A and 4B show micrographs of transfected cells and a graph representing transcription levels of transfected cells. FIG. 4A, shows photomicrographs of CH3T10½ cells transfected with Flag—GLI and either Myc—SUFU-wt or Myc-SUFU-Δex8, and stained for immunofluorescence with monoclonal antibody against Flag and polyclonal antibody against Myc followed (as secondary antibodies) by rhodamine-labeled antibody against rabbit and FITC-labelled antibody against mouse IgG. Cells were also stained with DAPI for visualization of the nucleus. FIG. 4B, shows CH3T10½ cells transfected with a GLI-responsive promoter construct (8*GLI) and various doses of GLI, and either the empty pcDNA 3,1 (+) vector (control), SUFU-wt or SUFU-Δex8. SUFU-wt strongly inhibited transcription promoted by GLI at very low dosages, whereas SUFU-Δex8 had no effect as compared with the control. Very high doses of SUFU-Δex8 blocked transcription from the 8*GLI reporter to a moderate degree, but not nearly as much as SUFU-wt. Error bars, ±s.e.m.

### DETAILED DESCRIPTION OF THE INVENTION

The present invention relates to the identification of the SUFU gene as a tumor suppressor gene involved in medulloblastoma. Mutations in the human SUFU gene have now been identified and associated with medulloblastoma. This knowledge now enables diagnostic methods for detecting alterations in the wild type SUFU gene for the diagnosis, prognosis and identification of subjects having or at risk for developing medulloblastoma. This also enables methods for the treatment of medulloblastoma and the screening of candidate drug compounds for their ability to block SHH signalling and restore SUFU function.

The invention in general, enables the diagnosis of cancers involving a mutation in the SUFU gene. In aspects, this includes inappropriate SHH signalling where SUFU is required to control such signalling. Such cancers may include but are not limited to medulloblastoma, nevoid basal cell carcinoma (NBCCS), basal cell carcinoma (BCC), meningioma, colon cancer, muscle tumors and sarcomas.

Both germline and somatic SUFU mutations have been identified accompanied by loss of heterozygosity (LOH) of the wild type allele. Several of the observed mutant genes encode truncated proteins which are unable to export transcription factors of the GLI family from nucleus to cytoplasm, resulting in an increase in SHH signaling.

The mutations of the SUFU gene and predicted protein are shown in Table 1 and summarized as follows: IVS8+1G→A; IVS1-1G→T; 1129delTCCGGAG; IVS1-1A→T; E1 143insA; and a 2.5-2.8 Mb deletion on chromosome 10q. Also identified were two missense mutations identified as likely polymorphisms in Table 1: C44T and G118T. A further mutation of SUFU(212-484), a N-terminal deletion was also made and resulted in the expression of a mutant non-functional protein.

Identification of SUFU Gene Mutations

Frequent loss of heterozygosity (LOH) on chromosome 10q24 in medulloblastomas suggests that this region contains one or more tumor-suppressor genes (20). SUFU was mapped to chromosome 10q24.3 by fluorescence in situ hybridization (FISH). Radiation-hybrid mapping of SUFU and BTRC (encoding a ubiquitin ligase involved in both SHH and Wnt signaling) showed an identical map location for both genes at chromosome 10q24.3 (data not shown) distal to the tumor-suppressor gene PTEN on chromosome 10q23.31. Human genome sequence data indicated that BTRC is approximately 1 megabase (Mb) centromeric to SUFU. Screening the Roswell Park human genomic bacterial artificial chromosome (BAC) library with a SUFU cDNA probe led to the identification of two BACs containing genomic SUFU: 2F13 and 124G18. Subcloning and sequence analysis of these BACs revealed that SUFU has 12 exons shown in Table 2.

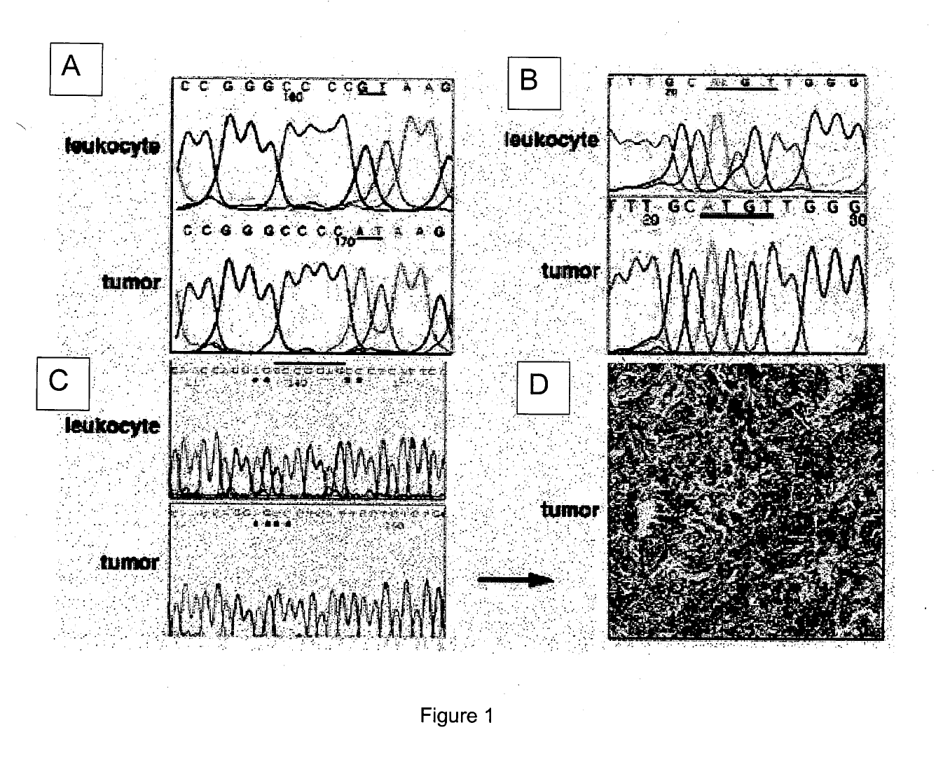

Mutational analyses of PTEN [phosphatase and tensin homologue deleted in chromosome 10] and BTRC [beta-transducing repeat-containing protein gene] indicated no mutations in two series of 21 and 36 sporadic medulloblastomas, respectively. Truncating mutations of SUFU were identified in 4 of 46 samples (9%) of medulloblastoma by sequence analysis of exons 1-12 and their surrounding intronic sequences. This mutation frequency is comparable to that observed for PTCH (9%) and CTNNB1 (encoding β-catenin; 5% (7,21). Sequence analysis and RT-PCR analyses revealed that these four SUFU mutations predicted truncated protein products (FIG. 1 and Table 1). Two missense mutations were also identified, the nonconservative P15L near the amino terminus and the relatively conservative A340S (Table 1). All four truncating mutations and the P15L mutation were accompanied by LOH and/or loss of the wild type allele (FIG. 1A-1C and Table 1), but no LOH was detected of the A340S variant. All four medulloblastomas with SUFU truncating mutations were of the desmoplastic subtype (FIG. 1D and Table 1). Desmoplastic tumors make up about 20-30% of medulloblastomas and have a more nodular architecture than ‘classical’ medulloblastoma and may have a better prognosis. Activation of the SHH pathway is particularly high in desmoplastic medulloblastomas, as shown by increased expression of the SHH target genes GLI, SMOH and PTCH12. Based on these experimental results it was concluded that SUFU functions as a tumor-suppressor gene in at least a subset of desmoplastic medulloblastomas.

The individual with a medulloblastoma bearing the IVS8+1G→A intron 8 truncating mutation was a 4-year-old boy with some phenotypic characteristics suggestive of NBCCS, including frontal bossing, prominent jaw and hypertelorism but did not exhibit odontogenic cysts, falx calcification, skin pits or BCCs (these characteristics may not arise until later in life in people with NBCCS). This child also had severe developmental delay: he was unable to walk and speak, and did not recognize his parents as his primary care-givers. Although some degree of cognitive impairment may follow treatment for medulloblastoma, the child's examination and history were more in keeping with a developmental syndrome. Neither parent had any evidence of NBCCS and there was no family history of cancer.

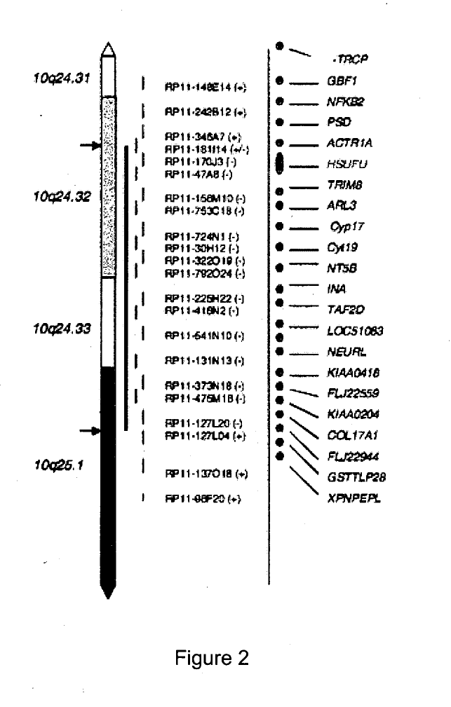

The IVS8+1G→A mutation detected in this boy's medulloblastoma was not present in his germline DNA. Southern blotting using an SUFU cDNA probe revealed no differences in the child's DNA compared with controls (data not shown). Metaphase FISH was carried out using a BAC clone encompassing SUFU in its entirety on lymphoblastoid cell lines from both parents and child. Only one chromosome 10q24 signal was observed for the child but two signals for each parent. FISH of the child's peripheral blood lymphocytes confirmed this finding. Haplotyping indicated that part of the paternal chromosome was lost in the child. Additional FISH experiments using contiguous BAC clones in the region of chromosome 10q24.3 showed that the child had a deletion of 2.5-2.8 Mb that encompassed chromosomal sub-bands 10q23.32-10q25.1, including at least 28 genes but sparing the centromeric BTRC (FIG. 2). Some individuals with cytogenetic interstitial deletions of 10q22-10q26 share some of the phenotypic characteristics observed in the child with the somatic IVS8+1G→A mutation, including psychomotor retardation, hypertelorism and a broad nasal bridge (23). In this child, loss of contiguous genes at 10q-including SUFU-was therefore associated with both medulloblastoma and a NBCCS-like phenotype with profound developmental delay.

Peripheral blood DNA was sequenced from individuals with medulloblastomas bearing SUFU mutations and demonstrated that the exon 2 splice-site mutation (IVS1-1A→T) and the exon 1 insertion (143insA) were present in lymphocyte DNA. In each case, the wild type allele was either deleted or mutated in the corresponding tumor (Table 1). The individual with the IVS1-1A→T mutation was adopted, and his biological family history was unavailable. The individual with the 143insA mutation (Table 1) had no known family history of any cancer. Six years after radiotherapy for his medulloblastoma, however, he developed a meningioma in the region of the radiation field. These two individuals with heterozygous germline point mutations of SUFU had no discernible developmental abnormalities; this is lo consistent with the very subtle phenotypic alterations seen in Drosophila homozygous SUFU mutants.

To study the functional consequences of the SUFU truncation mutants, the SUFU IVS8+1G→A variant was employed. The corresponding protein lacks the carboxy-terminal half of SUFU (designated SUFU-Δex8). A corresponding N-terminal deletion mutant protein, designated SUFU(212-484)-Myc was constructed. Transfection of epitope-tagged wild type SUFU, Δex8 or Δ-212 mutants together with either GLI or GLI2 showed that wild type SUFU can bind GLI and GLI2, whereas the Δex8 and Δ-212 mutants cannot (FIG. 3A-3C). The distribution of SUFU and GLI in the nucleus and cytoplasm was studied by transfecting expression vectors bearing either wild type Δex8 mutant SUFU together with GLI into C3HT10½ cells. Fluorescence microscopy showed that GLI transfected by itself was predominantly localized in the nucleus. Wild type SUFU was primarily found in the cytoplasm and, in its presence, GLI was also localized in the cytoplasm and hence nonactive (FIG. 4A). In contrast, the Δex8 mutant localized in the nucleus, and in its presence GLI accumulated substantially in the nucleus in all of the cells observed (FIG. 4A). Administration of leptomycin B, a CRM-1-dependent inhibitor of nuclear export, blocked the effects of wild type SUFU so that both wild type SUFU and GLI accumulated in the nucleus (FIG. 4A). As predicted, leptomycin B has no effect on SUFU-Δex8 and GLI cotransfectants, as both proteins were already localized in the nucleus (FIG. 4A). Finally, transfection of wild type SUFU repressed transcriptional activation by GLI from a GLI-responsive promoter (8*3′GLI-BS-Luc) with very small amounts of input DNA, whereas transfection of SUFU-Δex8 produced results no different from transfection of an empty vector control (FIG. 4B). We deduced that the SUFU-Δex8 mutant protein does not block GLI-mediated transcriptional activation at physiological doses (24).

The SUFU protein can repress Wnt signaling by binding β-catenin and exporting it from the nucleus (25). It is demonstrated that the medulloblastoma-derived SUFU-Δex8 mutant does not suppress Wnt signaling (data not shown). The present data supports a model in which the tumor-derived SUFU-Δex8 is unable to bind GLI transcription factors and export them from the nucleus, resulting in activation of SHH target genes. This is a new mechanism of tumor suppression that entails modulation of the nuclear-cytoplasmic shuttling of transcription factors.

Identification of Further Mutations

The identification of the SUFU mutations as described herein may now lead to the identification of further mutations in the SUFU gene leading to medulloblastoma and associated cancer development and diagnosis and thus are within the scope of the present invention. Mutated SUFU gene or gene products as described herein are characterized by premature stop codons, deletions, insertions or change of particular amino acids. Premature stop codons and deletions are detected by decreased size of the gene or gene product (mRNA transcript or cDNA). Similarly, insertions can be detected by increased size of the gene or gene product. Alternatively, mutations can be determined by sequencing of the gene or gene product according to standard methods. Such methods have been used herein to identify the noted mutations.

Amplification assays and hybridization probes can be selected to specifically target particular abnormalities. For example, where the abnormality is a deletion, nucleic acid probes or amplification primers can be selected that specifically hybridize to or amplify, respectively, the nucleic acid sequence that is deleted in the abnormal gene. The probe will fail to hybridize, or the amplification reaction will fail to provide specific amplification, to abnormal versions of the suppressor of fused nucleic acids which have the deletion. Alternatively, the probe or amplification reaction can be designed to 5 span the entire deletion or either end of the deletion (deletion junction). Similarly, probes and amplification primers can be selected that specifically, target point mutations or insertions. Methods for detecting specific mutations are described in, for example, U.S. Pat. No. 5,512,441, the entire contents of which are incorporated by reference herein. In the case of PCR amplification primers can be designed to hybridize to a portion of the suppressor of fused gene but the terminal nucleotide at the 3′ end of the primer can be used to discriminate between the mutant and wild-type forms of the SUFU gene. If the terminal base matches the point mutation or the wild-type sequence, polymerase dependent extension can proceed and an amplification product is detected (Sommer et al., (1989) Mayo Clin. Proc. 64:1361-1372). By using appropriate controls, one can develop a kit having both positive and negative amplification products. The products can be detected using specific probes or by simply detecting their presence or absence. A variation of the PCR method uses LCR where the point of discrimination, i.e., either the point mutation or the wild-type bases fall between the LCR oligonucleotides. The ligation of the oligonucleotides becomes the means for discriminating between the mutant and wild-type forms of the suppressor of fused gene.

A variety of automated solid-phase detection techniques are also appropriate for detecting the presence or absence of further mutations in the SUFU gene. For instance, very large scale immobilized polymer arrays (VLSIPS™ available from Affymetrix, Inc. Santa Clara, Calif.) are used for the detection of nucleic acids having specific sequences of interest (Fodor et al. (1991) Science, 251: 767-777; Sheldon et al. (1993) Clinical Chemistry 39(4): 718-719. and Kozal et al. (1996) Natural Medicine 2(7): 753-759). Further methods for detecting mutations are also described in, for example, Tijssen (1993) “Laboratory Techniques in biochemistry and molecular biology, hybridization with nucleic acid probes parts I and II,” Elsevier, New York, and Choo (ed) (1994) Methods In Molecular Biology Volume 33-In situ Hybridization Protocols, Humana Press Inc., New Jersey.

Transcription levels (and thereby expression) of a mutated SUFU gene may be assessed in a sample, the nucleic acid sample is one in which the concentration of the mRNA transcript(s) of the suppressor of fused gene, or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that lo the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Where more precise quantification is required appropriate controls can be run to collect for variations introduced in sample preparation and hybridization as described herein. In addition, serial dilutions of “standard” target mRNAs can be used to prepare calibration curves according to methods well known to those of skill in the art. Of course, where simple detection of the presence or absence of a transcript is desired, no elaborate control or calibration is required.

The expression of the human SUFU gene, both normal wild type and mutated (where the mutation leads to a protein product) can also be detected and/or quantified by detecting or quantifying the expressed SUFU polypeptide. SUFU polypeptides can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitating reactions, immunodiffusion (single or double), immunoelectrophoresis, radioimmunoassay(RIA), enzyme-linked immunosorbent assays (ELISAs), immunofluorescence assays, Western blotting, and the like. In embodiments, the SUFU wild type or mutant polypeptides may be detected using gel electrophoresis or detected using an immunoassay. The immunoassay is being characterized by detection of specific binding of a SUFU polypeptide to SUFU fused antibody as opposed to the use of other physical or chemical properties to isolate, target, and quantify the analyte. The presence or absence of a SUFU polypeptide in a biological sample can be determined using electrophoretic methods and may indicate the presence of a nucleic acid deletion leading to the absence of protein expression. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

Diagnostic Methods

The present invention now enables diagnostic methods for detecting alterations of the wild type human SUFU gene and human SUFU cDNA, such alterations indicating a predisposition to medulloblastoma and related tumors or, in a subject presenting with a brain tumor, provide a means of differential diagnosis of medulloblastoma. Medulloblastoma is commonly referred to as a primitive neuroectodermal tumor also referred to as an undifferentiated neuroectodermal tumor of the cerebellum. As such, the present invention would provide for the diagnosis of any medulloblastoma or related tumor that involves a SUFU mutation. A “mutation” as herein described would lead to a medulloblastoma phenotype. Furthermore, the invention encompasses the diagnosis of tumors which show disruption of the SHH signaling pathway and may be amenable to treatment by the methods and pharmaceutical compositions described herein and include skin cancers such as basal cell carcinoma, colon cancers, muscle tumors and sarcomas.

Members of a family with a history of medulloblastoma, or other SUFU mutation-associated tumors, may now be screened for alterations/mutations from normal in the SUFU gene, alterations indicating a possible predisposition to tumor development or that tumor development is in the early stage should the subject be otherwise asymptomatic. Those members in whom a gene alteration is detected may then be kept under careful scrutiny, so that early tumor detection can facilitate early treatment. Screening may be carried out prenatally, on a fetus.

Where a subject already shows tumor development, examination of tumor and other tissues allows the identification of a germline mutation and possible predisposition to further tumor development, again requiring careful follow up.

Detection of alterations from the wild type SUFU gene may be detected by many different methods known to those of skill in the art. The cDNA sequence of the SUFU gene has been described (GenBank Accession Number AY081818). The exon structure of human SUFU and flanking intron sequences are shown in Table 2.

“Alterations” of the wild type SUFU gene include mutations of the gene, including deletions, insertions, inversions or point mutations, either in the regulatory regions or the coding regions of the gene. Testing for alterations may be carried out on DNA or RNA from a biological sample such as blood, tissue biopsies or tumor biopsies obtained from the subject to be tested.

In one embodiment, the invention provides a method for determining whether a subject is at risk for development or has a tumor associated with an alteration of the SUFU gene, for example a medulloblastoma, the method comprising the steps of:
(a) obtaining a nucleic acid sample from the subject; and
(b) conducting an assay on the nucleic acid sample to determine the presence or absence of a SUFU gene mutation associated with tumor development, wherein the presence of such a SUFU gene mutation indicates that the subject is at risk for development of a tumor.

In other embodiments of the invention is a method for the diagnosis of a tumor associated with a mutation in a SUFU gene, the method comprising: detecting in the sample from a subject suspected of having a tumor, a mutation in the SUFU gene or a protein encoded thereby, wherein detection of mutation in the gene or the protein encoded thereby is indicative of having the tumor.

As described herein, mutations of the SUFU gene can be detected using different assays, for example, by sequencing exons and introns of the gene, restriction fragment length polymorphisms (RFLP) analysis, PCR-RFLP analysis, allele-specific hybridizations, mutation specific polymerase chain reactions (MSPCR), or by single stranded conformational polymorphism (SSCP) detection. Many suitable assays are known to those of skill in the art and described for example in U.S. Pat. Nos. 6,475,723, 6,361,949, and 6,558,013 (the disclosures of which are herein incorporated by reference in their entirety). The nucleic acid may be RNA or DNA, for example mRNA or genomic DNA. Where the method is used to determine risk for medulloblastoma, the SUFU mutations listed in Table 1, for example, are indicative of a subject at risk for development of medulloblastoma.

The SUFU gene may, for example, be analysed for mutations as described herein.

Therapeutic Methods

The invention also enables therapeutic methods and compositions for the treatment of medulloblastoma or another tumor associated with a SUFU mutation, in a mammal, including a human.

Methods of treatment in accordance with the invention are aimed at restoring normal SUFU function in cells in which a mutation is disrupting function, for example in medulloblastoma cells in a subject suffering from a SUFU mutation-associated medulloblastoma.

Such methods include gene therapy to restore normal function to the cells at the gene level and delivery of normal SUFU protein to circumvent the effects of the malfunctioning gene. Gene therapy may, for example, involve administration to the subject of a construct comprising an expression vector containing a nucleotide sequence encoding a wild type SUFU protein. Suitable expression vectors include retroviral, adenoviral and vaccinia virus vectors. Administration may be intravenous, oral, subcutaneous, intramuscular, intraperitoneal or directly into the tumor or its supplying blood vessels.

A large number of gene delivery methods are well known to those of skill in the art and may include, for example liposome-based gene delivery (Debs and Zhu (1993) WO 93/24640; Mannino and Gould-Fogerite (1988) BioTechniques 6(7): 682-691; Rose U.S. Pat No. 5,279,833; Brigham (1991) WO 91/06309; and Felgner et al. (1987) Proc. Natl. Acad Sci. USA 84: 7413-7414), and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4:43, and Cornetta et al. Hum. Gene Ther. 2:215 (1991)). Widely used retroviral vectors include those based upon murine leukemia virus (MuLV), gibbon ape leukemia virus (GaLV), Simian Immuno deficiency virus (SIV), human immuno deficiency virus (H IV), and combinations thereof. See, e.g., Buchscher et al. (1992) J. Virol. 66(5) 2731-2739; Johann et al. (1992) J. Virol. 66 (5):1635-1640 (1992); Sommerfelt et al., (1990) Virology 176:58-59; Wilson et al. (1989) J. Virol. 63:2374-2378; Miller et al., J. Virol. 65:2220-2224 (1991 ); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra).

AAV-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invest. 94:1351 and Samulski (supra) for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin et al. (1984) Mol. Cell. Biol. 4:2072-2081; Hermonat and Muzyczka (1984) Proc. Natl. Acad. Sci. USA 81:6466-6470; McLaughlin et al. (1988) and Samulski et al. (1989) J. Virol. 63:03822-3828. Cell lines that can be transformed by rAAV include those described in Lebkowski et al. (1988) Mol. Cell. Biol. 8: 3988-3996.

In a further embodiment of the invention there is provided therapy through removal or blocking of the mutant gene product, as well as supplementation with the normal gene product by amplification, by genetic and recombinant techniques or by immunotherapy. Correction or modification of the defective gene product by protein treatment immunotherapy (using antibodies to the defective protein) or knock-out of the mutated gene is within the scope of the invention.

For immunotherapy and isolated of the SUFU protein, there are available many methods of making antibodies which are known to persons of skill in the art. A number of immunogens may be used to produce antibodies specifically reactive with SUFU polypeptides. Recombinant or synthetic polypeptides of 10 amino acids in length, or greater, selected from amino acid sub-sequences of the human sequence of Table 2 are the preferred polypeptide immunogen (antigen) for the production of monoclonal or polyclonal antibodies. In one class of preferred embodiments, an immunogenic peptide conjugate is also included as an immunogen. Naturally occurring polypeptides are also used either in pure or impure form. Recombinant polypeptides are expressed in eukaryotic or prokaryotic cells (as described below) and purified using standard techniques. The polypeptide, or a synthetic version thereof, is then injected into an animal capable of producing antibodies. Either monoclonal or polyclonal antibodies can be generated for subsequent use in immunoassays to measure the presence and quantity of the polypeptide.

Methods of producing polyclonal antibodies are known to those of skill in the art. In brief, an immunogen (antigen), preferably a purified polypeptide, a polypeptide coupled to an appropriate carrier (e.g., GST, keyhole limpet hemocyanin, etc.), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired (see, e.g., Coligan (1991) Current Protocols in Immunology Wiley/Greene, NY; and Harlow and Lane (1989) Antibodies: A Laboratory Manual Cold Spring Harbor Press, NY).

Antibodies, including binding fragments and single chain recombinant versions thereof, against predetermined fragments of SUFU polypeptides are raised by immunizing animals, e.g., with conjugates of the fragments with carrier proteins as described above. Typically, the immunogen of interest is a peptide of at least about 5 amino acids, more typically the peptide is 10 amino acids in length, preferably, the fragment is 15 amino acids in length and more preferably the fragment is 20 amino acids in length or greater. The peptides are typically coupled to a carrier protein (e.g., as a fusion protein), or are recombinantly expressed in an immunization vector. Antigenic determinants on peptides to which antibodies bind are typically 3 to 10 amino acids in length.

Monoclonal antibodies are prepared from cells secreting the desired antibody. These antibodies are screened for binding to normal or modified polypeptides, or screened for agonistic or antagonistic activity, e.g., activity mediated through a SUFU protein. In some instances, it is desirable to prepare monoclonal antibodies from various mammalian hosts, such as mice, rodents, primates, humans, etc. Description of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, supra; Goding (1986) Monoclonal Antibodies: Principles and Practice (2d ed.) Academic Press, New York, N.Y.; and Kohler and Milstein (1975) Nature 256: 495-497. The polypeptides and antibodies of the present invention are used with or without modification, and include chimeric antibodies such as humanized murine antibodies. Other suitable techniques involve selection of libraries of recombinant antibodies in phage or similar vectors (see, e.g., Huse et al. (1989) Science 246: 1275-1281; and Ward et al. (1989) Nature 341: 544-546; and Vaughan et al. (1996) Nature Biotechnology, 14: 309-314).

Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionucleotides, enzymes, substrates, cofactors, inhibitors, fluorescence moieties, chemiluminescence moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced. See, Cabilly, U.S. Pat. No. 4,816,567; and Queen et al. (1989) Proc. Nat'l Acad. Sci. USA 86: 10029-10033. Antibodies specific for a SUFU protein are also used for affinity chromatography in isolating SUFU polypeptides. Columns are prepared, e.g., with the antibodies linked to a solid support, e.g., particles, such as agarose, Sephadex, or the like, where a cell lysate is passed through the column, washed, and treated with increasing concentrations of a mild denaturant, whereby purified SUFU polypeptides are released.

The antibodies can be used to screen expression libraries for particular expression products such as normal or abnormal human SUFU protein. Usually the antibodies in such a procedure are labeled with a moiety allowing easy detection of presence of antigen by antibody binding. Antibodies raised against SUFU polypeptides can also be used to raise antiidiotypic antibodies. These are useful for detecting or diagnosing various pathological conditions related to the presence of the respective antigens.

The anti-SUFU antibodies can be administered to an organism (e.g., a human patient) for therapeutic purposes (e.g., to block the action a non-functional mutated SUFU polypeptide or as targeting molecules when conjugated or fused to effector molecules such as labels, cytotoxins, enzymes, growth factors, drugs, etc.). Antibodies administered to an organism other than the species in which they are raised are often immunogenic. Thus, for example, murine antibodies administered to a human often induce an immunologic response against the antibody (e.g., the human anti-mouse antibody (HAMA) response) on multiple administrations. The immunogenic properties of the antibody are reduced by altering portions, or all, of the antibody into characteristically human sequences thereby producing chimeric or human antibodies, respectively. Methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369).

A further method of treatment for a subject determined to have medulloblastoma includes method of RNAi (RNA interference) that may be used to inhibit the expression of the mutant SUFU gene and this may be done in conjunction with administration of normal SUFU gene by gene therapy or administration of normal SUFU protein. RNAi, RNA interference, is a mechanism of post-transcriptional gene silencing. Specific gene silencing is mediated by short strands of duplex RNA of approximately 21 nucleotides in length (termed small interfering RNA or siRNA) that target the cognate mRNA sequence for degradation. This technique is relatively simple, giving rise to an knock down phenotype that can be confirmed with many antibody based detection systems (such as ELISA or Western Blotting), or if an antibody is not available, by RT-PCR or functional assays. The siRNA can be administered alone as a composition and targeted to a mutated SUFU gene. The siRNA may also be contained within a plasmid or vector that results in the production of the siRNA. Methods for making siRNA and cell transformation are described for example in U.S. patent application 2002/0173478, U.S. patent application 2002/0162126, PCT/US01/10188, PCT/EP01/13968 and in Simeoni et al., 2003 Nucleic Acids Res June 1;31(11):2717-24 (the disclosures of which are incorporated herein in their entirety). Methods for making siRNA plasmids or vectors are also known and described for example in U.S. patent application 2003/0104401, in Morris et al., 1997. Nucleic Acid Res. July 15:25(14)-2730-6 and in Van De Wetering et al., 2003, EMBO J. June;4(6):609-15 (the disclosures of which are incorporated herein in their entirety). Suitable lipid-based vectors may include but are not limited to lipofectamine, lipofectin, oligofectamine and GenePorter™.

In a further embodiment of the invention, treatment of medulloblastoma or a tumor involving inappropriate SHH signalling can be performed by replacing the mutant protein with normal protein, or by modulating the function of the mutant protein. It may also be possible to modify the pathophysiologic pathway (e.g., a signal transduction pathway) in which the protein participates in order to correct the physiological defect. To replace the mutant protein with normal protein, or with a protein bearing a deliberate counterbalancing mutation it is necessary to obtain large amounts of pure SUFU protein from cultured cell systems which can express the protein. Delivery of the protein to the affected brain areas or other tissues can then be accomplished using appropriate packaging or administrating systems.

SUFU protein, or an active portion thereof, may be prepared by conventional recombinant methods, using the cDNA sequence of ATCC Accession Number AY081818 or a selected portion thereof and used in therapeutic methods to try to provide adequate SUFU functioning such that SHH signalling is not abnormal leading to tumor formation. A pharmaceutical composition according to the invention will include a therapeutically effective amount of the wild-type SUFU protein with a carrier. A therapeutically effective amount is considered that amount which, when administered to a subject, provides a therapeutic benefit to the patient.

Methods of treatment may also involve administration of a therapeutic composition comprising a compound which can block SHH signaling. Such compounds have been described and include, for example, the steroid alkaloid cyclopamine (Cooper et al. (1998) Science 280:1603-7). The SUFU polypeptides, SUFU polypeptide subsequences, anti-SUFU antibodies, and anti-SUFU antibody-effector (e.g., enzyme, toxin, hormone, growth factor, drug, etc.) conjugates or fusion proteins may be used for parenteral, topical, oral, or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that the SUFU polypeptides when administered orally, should be protected from digestion. This is typically accomplished either by complexing the protein with a composition to render it resistant to acidic and enzymatic hydrolysis or by packaging the protein in an appropriately resistant carrier such as a liposome. Means of protecting proteins from digestion are well known in the art.

The pharmaceutical compositions of this invention are particularly useful for administration to treat tumors associated with abnormal SUFU gene functioning. In another embodiment, the compositions are useful for parenteral administration, such as intravenous administration or administration into a body cavity or lumen of an organ. The compositions for administration will commonly comprise a solution of the SUFU polypeptide, antibody or antibody chimera/fusion dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of chimeric molecule in these formulations can vary widely, and will be selected primarily based on fluid volumes, viscosities, body weight and the like in accordance with the particular mode of administration selected and the patient's needs.

Typical pharmaceutical compositions for intravenous administration may be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

Compositions containing the SUFU polypeptides, antibodies or antibody chimer/fusions, or a cocktail thereof (i.e., with other proteins), can be administered for therapeutic treatments. In therapeutic applications, compositions are administered to a patient suffering from a disease (e.g., medulloblastoma) in an amount sufficient to cure or at least partially arrest the disease and its complications. An amount adequate to accomplish this is lo defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the proteins of this invention to effectively treat the patient.

Screening for Candidate Drug Compounds

The invention further enables a method of screening candidate drug compounds for their ability to block SHH signaling and restore normal SUFU function. Compounds shown to have the ability to block SHH signaling and restore normal SUFU function are candidate pharmaceuticals for the treatment of medulloblastoma and other tumors associated with an altered SUFU gene. Suitable methods for screening candidate compounds are described for example in U.S. Pat. No. 6,413,717 (the disclosure of which is incorporated herein in its entirety).

Compounds that may prevent excessive GLI-induced transcription through activation of SUFU or by aiding the sequestering of SUFU and GLI from the nucleus can be tested in assays such as those described herein. The inventors transfected C3H10T½ cells with plasmid DNA containing wild type or Δex8 mutant together with GLI. Immunofluorescence was carried out as described herein. Also examined was transcription using cells similarly transfected as above but including the reporter gene GLI-BS-Δ51LucII.

The immunofluorescence assay localizes GLI and SUFU whereas the promoter assay indicates the effects of SUFU (wild type or mutant) on GLI-induced transcription.

Localization of SUFU and/or GLI may be undertaken before and after drug treatment. Functional significance of the drug may be tested using the promoter assay. Decreased transcription in the cell culture model using expression of the mutated SUFU indicates a candidate compound with therapeutic potential.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Tumor Samples and Isolation of Nucleic Acids

Samples of pediatric medulloblastoma under the guidelines of the Hospital for Sick Children Research Ethics Board were collected, flash-frozen directly after surgical removal and stored the samples in liquid nitrogen. Additional tumor samples came from the Brain Tumor Tissue Bank (London, Ontario, Canada), the Pediatric Co-operative Human Tissue Network of the US National Cancer Institute (Bethesda, Md.), the Tissue Bank of the Brain Tumor Research Center at UCSF (San Francisco, USA) and the Children's National Medical Center (Washington, USA). Individuals with NBCCS but no mutations in PTCH were identified in a National Cancer Institute study (6,26) or at the University of Queensland (Australia) (27). In every case, affected individuals or their guardians provided informed consent and/or took part in protocols approved by local or national institutional review boards.

Total RNA was isolated using Trizol reagent (Gibco-BRL), and genomic DNA was isolated by overnight digestion in proteinase K followed by standard phenol/chloroform extraction. After dissecting tumor tissue from paraffin slides and preparing DNA by xylene extraction, digestion was carried out in proteinase K at 55° C. overnight.

### Example 2

### Determination of Genomic Structure and Chromosomal Location of SUFU and BTRC (Beta-Transducin Repeat-Containing Protein Gene)

Using SUFU cDNA as a probe, clones 124G18 and 2F13 were isolated from the Roswell Park Cancer Institute human genomic BAC library, and subclones sequenced to determine intron-exon boundaries. PCR was used to screen a BAC library specific for chromosome 10q (Genome Systems) for clones containing human BTRC and carried out radiation hybrid mapping of SUFU and BTRC with the GB4 panel (Research Genetics). All primer sequences are available on request.

cDNAs were generated using SuperScript II RT (Gibco-BRL) with both oligo-dt and SUFU-specific primers. Two nested PCRs were performed to amplify exons 1-4 and exons 4-12, respectively, the PCR products were subcloned with a TA cloning Kit (Invitrogen) and sequenced using the Cy-5/5.5-labeled M13 primer.

### Example 3

### Fluorescence in situ Hybridization (FISH)

Lymphocytes were cultured from peripheral blood or cell lines infected with Epstein—Barr virus and incubated the cells for 30 min with colcemid (0.1 μg/ml) before harvesting. A contiguous series of BAC clones from chromosome 10q24-q25 was labelled with biotin using the BioNick Labeling system, and BAC RP11-59D04 on chromosome 10p15 was labelled with digoxigenin as a control. FISH signals were detected with fluorescein-avidin D (Vector) and fluorescein-labeled antibody against avidin D (Vector) for biotin-labeled probes, and with antibody against digoxigenin, digoxigenin-labeled antibody against mouse Ig, and rhodamine-labeled antibody against digoxigenin (Boehringer Mannheim) for digoxigenin-labeled probes.

### Example 4

### Mutational Analysis

Using intronic primers designed to include an 5′-M13 sequencing cassette for all 12 exons of SUFU, PCR reactions were carried out in a PTC-100 Programmable Thermal Controller for 35-40 cycles, usually at an annealing temperature of 60° C. In some cases, nested PCR was performed using a second set of intronic primers. Amplified products were sequenced as previously described (28). Primer sequences and protocols are available on request.

Using single-strand conformation polymorphism (SSCP) as previously described (29), 36 medulloblastomas were screened for each exon of BTRC and sequenced exons that migrated aberrantly through the SSCP gel (11 of 38). LOH was measured of the markers D10S215, D10S520 and D10S540 near PTEN (phosphatase and tensin homologue deleted in chromosome 10) in 21 medulloblastomas and sequenced genomic DNA for all coding exons of PTEN from the corresponding tumors (29).

### Example 5

### SUFU Expression Constructs

SUFU cDNA was amplified from the Marathon fetal brain cDNA (Clonetech) into pTAdv (Clonetech), created an in-frame N-terminal Myc tag by PCR of the SUFU cDNA, and cloned the Myc-SUFU cDNA into pcDNA3.1 (Invitrogen). The mutant SUFU-Δex8 transcript (amplified from cDNA of medulloblastoma HMB2) was shuttled into the pcDNA3.1 wild type SUFU-Myc construct. The SUFU(212-484)-Myc expression vector was constructed by PCR and both strands of each expression construct were sequenced to rule out PCR errors. pCMV-Flag-GLI1 and pCMV-Flag-GLI2 have previously been described (30).

### Example 6

### Co-Precipitation Assay

293 cells were transfected with pCMV-Flag-GLI1 or pCMV-Flag-GLI2 and either pcDNA 3.1-Myc-SUFU, pcDNA 3.1-Myc-SUFU-Δex8 or pcDNA 3.1-Myc-SUFU(212484) using Superfect (Qiagen). Cells were lysed in 500 ml lysis buffer (50 mM HEPES, pH 7.5, 150 mM NaCl, 10% glycerol, 1% Triton X-100 and 1 mM EDTA), and the lysates incubated overnight at 4° C. with 2 μl monoclonal antibody against Myc (UBI), or 2 μl monoclonal antibody against Flag M2 (Sigma) and 50 μl 20% protein G-Sepharose beads (Sigma). After washing the beads five times in IP washing buffer (50 mM HEPES, pH 7.4, 150 mM NaCl, 100 μM ZnCl₂, 2 mM EDTA, 1% Nonidet P-40 and 10% glycerol), beads were re-suspended and boiled in Laemmli sample buffer. The samples were resolved by SDS-PAGE (immunoprecipitate/lysate ratio 50:1), and the gel transferred to polyvinylidene difluoride membranes (Immobilon-P) which were subsequently probed with monoclonal antibody against Flag-M2 (Sigma) or monoclonal antibody against Myc (UBI) and the signals visualized using enhanced chemiluminescence.

### Example 7

### Immunofluorescence

C3H10T½ cells were grown on glass cover slips and transfected with plasmid DNA using Fugene 6 (Roche). After 24 hours the cells were fixed in 4% paraformaldehyde at 37° C. for 10 minutes and rendered permeable by incubation in methanol for 2 minutes. The samples were incubated in blocking solution (PBS with 10% goat serum) for 1 hour at room temperature, with primary antibodies (rabbit antibody against Myc (Santa Cruz) and/or mouse antibody against Flag-M5 (Sigma)) for 1 hour at room temperature, and with secondary antibodies (FITC-labeled goat antibody against mouse IgG and rhodamine-labeled goat antibody against rabbit) overnight at 4° C. After counterstaining nuclei with DAPI, fluorescent images were acquired using a Leica DMIRE2 inverted fluorescence microscope with Open Lab software (Improvision).

### Example 8

### Promoter Assay

C3H10T½ cells were plated at a density of 5×10⁴cells/well in six-well plates and transfected them using Fugene (Roche) with the reporter gene 8*GLI-BS-Δ51 LucII (24), a reference plasmid pCMV-β-gal, appropriate expression constructs and sufficient pcDNA 3.1 to achieve an equal amount of DNA in each well. At 36 hours after transfection, cells were harvested and luciferase activity normalized with respect to β-galactosidase activity. All transfections were repeated in at least two independent experiments, which gave reproducible results.

GenBank accession numbers. The exons and surrounding intronic sequences for SUFU have been submitted to GenBank under the accession numbers AY081818, AY081819, AY081820, AY081821, AY081822, AY081823, AY081824, AY081825, AY081826, AY081827, AY081828 and AY081829.

Although preferred embodiments have been described herein in detail it is understood by those of skill in the art that using no more than routine experimentation, many equivalents to the specific embodiments of the invention described herein can be made. Such equivalents are intended to be encompassed by the scope of the claims appended hereto.

Numerous patents, patent publications and other publications are cited throughout this specification. The entire contents of each of these publications and patents are incorporated by reference herein as further written support for the teachings described in the paragraph where the patent and/or publication is cited.

### References

1. Dahmane, N. & Ruiz-i-Altaba, A. Sonic hedgehog regulates the growth and patterning of the cerebellum. Development 126, 3089-3100 (1999).

2. Wechsler-Reya, R. J. & Scott, M. P. Control of neuronal precursor proliferation in the cerebellum by Sonic Hedgehog. Neuron 22,103-114 (1999).

3. Hahn et al. Mutations of the human homolog of Drosophila patched in the nevoid basal cell carcinoma syndrome. Cell 85, 841-851 (1996).

4. Johnson et al. Human homolog of patched, a candidate gene for the basal cell nevus syndrome. Science 272, 1668-1671 (1996).

5. Gorlin, R. J. Nevoid basal-cell carcinoma syndrome. Medicine (Baltimore) 66, 98-113 (1987).

6. Kimonis et al. Clinical manifestations in 105 persons with nevoid basal cell carcinoma syndrome. Am. J. Med. Genet. 69, 299-308 (1997).

7. Raffel et al. Sporadic medulloblastomas contain PTCH mutations. Cancer Res. 57,842-845 (1997).

8. Unden et al. Mutations in the human homologue of Drosophila patched (PTCH) in basal cell carcinomas and the Gorlin syndrome: different in vivo mechanisms of PTCH inactivation. Cancer Res. 56,4562-4565 (1996).

9. Wolter, M., Reifenberger, J., Sommer, C., Ruzicka, T. & Reifenberger, G. Mutations in the human homologue of the Drosophila segment polarity gene patched (PTCH) in sporadic basal cell carcinomas of the skin and primitive neuroectodermal tumors of the central nervous system. Cancer Res. 57, 2581-2585 (1997).

10. Zurawel et al. Analysis of PTCH/SMO/SHH pathway genes in medulloblastoma. Genes Chromosom. Cancer 27, 44-51 (2000).

11. Taylor, M. D., Mainprize, T. G. & Rutka, J. T. Molecular insight into medulloblastoma and central nervous system primitive neuroectodermal tumor biology from hereditary syndromes: a review. Neurosurgery 47, 888-901 (2000).

12. Reifenberger et al. Missense mutations in SMOH in sporadic basal cell carcinomas of the skin and primitive neuroectodermal tumors of the central nervous system. Cancer Res. 58, 1798-1803 (1998).

13. Xie et al. Activating Smoothened mutations in sporadic basal-cell carcinoma. Nature 391, 90-92 (1998).

14. Kinzler et al. Identification of an amplified, highly expressed gene in a human glioma. Science 236, 70-73 (1987).

15. Goodrich, L. V., Milenkovic, L., Higgins, K. M. & Scott, M. P. Altered neural cell fates and medulloblastoma in mouse patched mutants. Science 277,1109-1113 (1997).

16. Hahn et al. Rhabdomyosarcomas and radiation hypersensitivity in a mouse model of Gorlin syndrome. Nature Med. 4, 619-622 (1998).

17. Nilsson et al. Induction of basal cell carcinomas and trichoepitheliomas in mice overexpressing GLI-1. Proc. Natl Acad. Sci. USA 97, 3438-3443. (2000).

18. Grachtchouk et al. Basal cell carcinomas in mice overexpressing Gli2 in skin. Nature Genet. 24, 216-217 (2000).

19. Oro et al. Basal cell carcinomas in mice overexpressing sonic hedgehog. Science 276, 817-821 (1997).

20. Bayani et al. Molecular cytogenetic analysis of medulloblastomas and supratentorial primitive neuroectodermal tumors by using conventional banding, comparative genomic hybridization, and spectral karyotyping. J. Neurosurg. 93, 437-448 (2000).

21. Zurawel, R. H., Chiappa, S. A., Allen, C. & Raffel, C. Sporadic medulloblastomas contain oncogenic beta-catenin mutations. Cancer Res. 58, 896-899 (1998).

22. Pomeroy et al. Prediction of central nervous system embryonal tumour outcome based on gene expression. Nature 415, 436-442 (2002).

23. Lobo, S., Cervenka, J., London, A. & Pierpont, M. E. Interstitial deletion of 10q: clinical features and literature review. Am. J. Med. Genet. 43, 701-703 (1992).

24. Sasaki, H., Hui, C., Nakafuku, M. & Kondoh, H. A binding site for Gli proteins is essential for HNF-3β floor plate enhancer activity in transgenics and can respond to SHH in vitro. Development 124, 1313-1322 (1997).

25. Meng et al. Suppressor of fused negatively regulates β-catenin signaling. J.Biol. Chem. 276, 40113-40119 (2001).

26. Chidambaram et al. Mutations in the human homologue of the Drosophila patched gene in Caucasian and African-American nevoid basal cell carcinoma syndrome patients. Cancer Res. 56, 4599-4601 (1996).

27. Wicking et al. Most germ-line mutations in the nevoid basal cell carcinoma syndrome lead to a premature termination of the PATCHED protein, and no genotype-phenotype correlations are evident. Am. J. Hum. Genet. 60, 21-26 (1997).

28. Liu et al. Mutation of the CDKN2A 5′ UTR creates an aberrant initiation codon and predisposes to melanoma. Nature Genet. 21, 128-132 (1999).

29. Raffel et al. Analysis of oncogene and tumor suppressor gene alterations in pediatric malignant astrocytomas reveals reduced survival for patients with PTEN mutations. Clin. Cancer Res. 5,4085-4090 (1999).

30. Ding et al. Mouse suppressor of fused is a negative regulator of sonic hedgehog signaling and alters the subcellular distribution of Gli1. Curr. Biol. 9, 1119-1122 (1999).

TABLE 1*
SUMMARY OF SUFU MUTATIONS IN MEDULLOBLASTOMAS AND CELL LINES

| | | | | Tumor or | LOH in | | Predicted | |
|---|---|---|---|---|---|---|---|---|
| Case | Phenotype | Morphology | Nucleotide | germline | tumor | RT-PCR | protein | Comments |
| 1 | NBCCS-like; | Desmoplastic | 2.5-Mb deletion | Germline | NA | ND | NA | Contiguous |
| | severe | | | | | | | deletion |
| | cognitive | | | | | | | syndrome in |
| | impairment | | | | | | | carrier |
| | | | IVS8 + 1G→A | Second hit in | Yes | E7 spliced | Frameshift at | Mutation |
| | | | splice donor | tumor | | to E9 | 5′ and | |
| | | | | | | | termination at | |
| | | | | | | | 3′ end of E9 | |
| 2 | MB | Desmoplastic | E1 143insA | Germline | Yes | ND | Frameshift | Mutation |
| | | | | | | | followed by | |
| | | | | | | | termination | |
| | | | | | | | codon | |
| | | | | | | | 60 bp 3′ to | |
| | | | | | | | mutation | |
| 3 | MB | Desmoplastic | 1129del | Tumor | Yes | Same as | Termination at | Mutation |
| | | | TCCGGAG | | | wild type | 5′ end of E10 | |
| 4 | MB | Desmoplastic | E2 splice | Germline | Yes | E1 spliced | Termination at | Mutation |
| | | | acceptor; IVS1 − 1A→T | | | to E4 | junction E1-E4 | |
| 5 | MB | NA | C44T | Tumor | Yes | Normal | P15L | Likely |
| | | | | | | | | polymorphism |
| 6 | MB | Desmoplastic | G1018T | Germline | No | Normal | A340S | Polymorphism |

MB, medulloblastoma;
NA, not applicable;
ND, not determine.

TABLE 2

| | |
|---|---|
| LOCUS | Y081918S01 623 bp DNA linear PRI |
| 04-APR-2002 | |
| DEFINITION | Homo sapiens suppressor of fused (SUFU) gene, exon 1. |
| ACCESSION | AY081818 |
| VERSION | AY081818 |
| KEYWORDS | . |
| SEGMENT | 1 of 12 |
| SOURCE | human. |
| ORGANISM | Homo sapiens |
| | Eukaryota; Metazoa; Chordata; Craniata; Vertebrata; |
| | Euteleostomi; |
| | Mammailia; Eutheria; Primates; Catarrhini; Hominidae; Homo. |
| REFERENCE | 1 (bases 1 to 623) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 623) |
| AUTHORS | Taylor, M. D., Hogg, D. and Rutka, J.T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..623 |
| | /organism=“Homo sapiens” |
| | /db_xref=“taxon:9606” |
| | /chromosome=“10” |
| | /map=“10q24” |
| exon | <305..486 |
| | /gene=“SUFU” |
| | /number=1 EXON 1 |
| BASE COUNT | 93 a 231 c 196 g 103 t |
| ORIGIN | |

| | | | | | |
|---|---|---|---|---|---|
| 1 | ctattgtcaa | gtcacacctt | ccctgcctgt | gactggcaca | gacattagcc |
| | aatgggtgct | | | | |
| 61 | tggatagggt | gcgccggcgc | cccgcccccc | ttagcgcccc | gccgccccga |
| | ggcaccctct | | | | |
| 121 | ggcagactcg | gcggcggcga | cagcctgggc | ggacagtgcg | ccgtgcgcag |
| | gcgcggagct | | | | |
| 181 | agacctcgct | gcagccccca | tcgcctcggg | gagtctcacc | caccgagtcc |
| | gcccgctggc | | | | |
| 241 | ccgtcagtgc | tctccccgtc | gtttgccctc | tccagttccc | ccagtgcctg |
| | ccctacgcac | | | | |
| 301 | cccgatggcg | gagctgcggc | ctagcggcgc | ccccggcccc | accgcgcccc |
| | cggcccctgg | | | | |
| 361 | cccgactgcc | cccccggcct | tcgcttcgct | ctttcccccg | ggactgcacg |
| | ccatctacgg | | | | |
| 421 | agagtgccgc | cgcctttacc | ctgaccagcc | gaacccgctc | caggttaccg |
| | ctatcgtcaa | | | | |
| 481 | gtactggtat | gctctgggcc | gcggggagac | ggacaggcgc | gggctggaaa |
| | gggttaaagc | | | | |
| 541 | gccgagggcg | aagtaatttg | tgggaggtgg | gaggaggggt | agagaatggt |
| | taaagcactc | | | | |
| 601 | agaaggaggg | cttcttgctg | cga | | |
| // | | | | | |

| | |
|---|---|
| LOCUS | Y081818S02 264 bp DNA linear PRI |
| 04-APR-2002 | |
| DEFINITION | Homo sapiens suppressor of fused (SUFU) gene, exon 2. |
| ACCESSION | AY081819 |
| VERSION | AY081819 |
| KEYWORDS | . |
| SEGMENT | 2 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 264) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wn= |
| | Signaling |
| JOURNAL | Unpublished |
| REFERENCE | 2 (bases 1 to 264) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..264 |
| | /organism=“Homo sapiens” |
| | /db_xref=“taxon:9606” |
| | /chromosome=“10” |
| | /map=“10q24” |
| exon | 77..211 EXON 2 |
| | /gene=“SUFU” |
| | /number=2 |
| BASE COUNT | 55 a 68 c 62 g 79 t |
| ORIGIN | |

| | | | | | |
|---|---|---|---|---|---|
| 1 | caaagtagag | cgccttagct | tgacattgtc | tgatttccag | gcttacacta |
| | acacccctgt | | | | |
| 61 | gttttgtttt | ttgcaggttg | ggtggcccag | accccttgga | ctatgttagc |
| | atgtacagga | | | | |
| 121 | atgtggggag | cccttctgct | aacatccccg | agcactggca | ctacatcagc |
| | ttcggcctga | | | | |
| 181 | gtgatctcta | tggtgacaac | agagtccatg | cgtgagtata | tgccacctgt |
| | tctttatcca | | | | |
| 241 | gagccttatt | cctgaggtct | tcct | | |
| // | | | | | |

| | |
|---|---|
| LOCUS | Y081818S03 275 bp DNA linear PRI |
| 04-APR-2002 | |
| DEFINITION | Homo sapiens-suppressor-of-fused. (SUFU) gene, exon 3. |
| ACCESSION | AY081820 |
| VERSION | AY081820 |
| KEYWORDS | . |
| SEGMENT | 3 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 275) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt. |
| | Signaling |
| JOURNAL | Unpublished |
| REFERENCE | 2 (bases 1 to 275) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..275 |
| | /organism=“Homo sapiens” |
| | /db_xref=“taxon:9606” |
| | /chromosome=“10” |
| | /map=“10g24” |
| exon | 79..215 |
| | /gene=“SUFU” Exon 3 |
| | /number=3 |
| BASE COUNT | 60 a 54 c 79 g 82 t |
| ORIGIN | |

| | | | | | |
|---|---|---|---|---|---|
| 1 | gggagaactt | tagactttca | agagagtgtt | tttcctaagg | taattgagct |
| | taaaacactt | | | | |
| 61 | gctttttatg | tctttcaggt | ttacaggaac | agatggacct | agtggttttg |
| | gctttgagtt | | | | |
| 121 | gacctttcgt | ctgaagagag | aaactgggga | gtctgcccca | ccaacatggc |
| | ccgcagagtt | | | | |
| 181 | aatgcagggc | ttggcacgat | acgtgttcca | gtcaggtagg | aggccagggc |
| | tggctgctgt | | | | |
| 241 | gctggtcctt | ttgccatgag | cctggttgac | tttga | |
| // | | | | | |

| | |
|---|---|
| LOCUS | Y081818S04 400 bp DNA linear PRI |
| 04-APR-2002 | |
| DEFINITION | Homo sapiens suppressor of fused (SUFU) gene, exon 4. |
| ACCESSION | AY081821 |
| VERSION | AY081821 |
| KEYWORDS | . |
| SEGMENT | 4 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 400) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 400) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..400 |
| | /organism=“Homo sapiens” |
| | /db_xref=“taxon:9606” |
| | /chromosome=“10” |
| | /map=“10q24” |
| exon | 95..237 |
| | /gene=“SUFU” EXON 4 |
| | /number=4 |
| BASE COUNT | 83 a 99 c 124 g 94 t |
| ORIGIN | |

| | | | | | |
|---|---|---|---|---|---|
| 1 | aagcctccca | gcctgggcta | gtgagatccc | agcccagatt | ccaggcctgg |
| | atctggggcc | | | | |
| 61 | ttgaacaatg | aggatccttg | tatctctccc | acagagaaca | ccttctgcag |
| | tggggaccat | | | | |
| 121 | gtgtcctggc | acagcccttt | ggataacagt | gagtcaagaa | ttcagcacat |
| | gctgctgaca | | | | |
| 181 | gaggacccac | agatgcagcc | cgtgcagaca | ccctttgggg | tagttacctc |
| | cctccaggtg | | | | |
| 241 | aggcacaggt | tggacgctgg | ctcaagcctt | cccgtgggaa | gggtcctggg |
| | aggacaagga | | | | |
| 301 | ggcttgagga | gggggagtga | agggagtggg | aggtttcctt | tggcctcgtc |
| | ctgatttctg | | | | |
| 361 | tttcctctga | tggtttgtca | ggtagattca | gatggtctga | |
| // | | | | | |

| | |
|---|---|
| LOCUS | Y081818S05 202 bp DNA linear PRI |
| 04-APR-2002 | |
| DEFINITION | Homo sapiens suppressor of fused (SUFU) gene, exon 5. |
| ACCESSION | AY081822 |
| VERSION | AY081822 |
| KEYWORDS | . |
| SEGMENT | 5 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 205) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 205) |
| AUTHORS | Taylor, M. D., Hogg, D. and Rutka, J. T. |
| TITLE | Direct Submassaon |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..205 |
| | /organism=“Homo sapiens” |
| | /db_xref=“taxon:9606” |
| | /chromosome=“10” |
| | /map=“10q24” |
| exon | 49..134 |
| | /gene=“SUFU” EXON 5 |
| | /number=“5” |
| BASE COUNT | 37 a 58 c 68 g 42 t |
| ORIGIN | |

| | | | | | |
|---|---|---|---|---|---|
| 1 | tggggggtgg | ccattaacac | acaatgggct | ttctatcctg | ggcctcagat |
| | cgttggtgtc | | | | |
| 61 | tgcactgaag | agctacactc | agcccagcag | tggaacgggc | agggcatcct |
| | ggagctgctg | | | | |
| 121 | cggacagtgc | ctatgtgagt | acccatgcaa | ggtgggagcg | cggctccctg |
| | ggcctggggg | | | | |
| 181 | tgggagtccc | tccactaccc | tccat | | |
| // | | | | | |

| | |
|---|---|
| LOCUS | Y081818S06 286 bp DNA linear PRI |
| 04-APR-2002 | |
| DEFINITION | Homo sapiens suppressor of fused (SUFU) gene, exon 6. |
| ACCESSION | AY081823 |
| VERSION | AY081823 |
| KEYWORDS | . |
| SEGMENT | 6 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 286) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 286) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..286 |
| | /organism=“Homo sapiens” |
| | /db_xref=“taxon:9606” |
| | /chromosome=“10” |
| | /map=“10q24” |
| exon | 123..195 |
| | /gene=“SUFU” EXON 6 |
| | /number=6 |
| BASE COUNT | 66 a 89 c 68 g 63 t |
| ORIGIN | |

| | | | | | |
|---|---|---|---|---|---|
| 1 | tccctgacca | cgaactattc | ccctgtgtcc | taggcctggg | gcagcaaaca |
| | gggcaggctg | | | | |
| 61 | taggcccagc | ccatcagccc | cagaccctca | gttaccattg | tatccccttt |
| | ccttgtccac | | | | |
| 121 | agtgctggcg | gcccctggct | gataactgac | atgcggaggg | gagagaccat |
| | atttgagatc | | | | |
| 181 | gatacacacc | tgcaagtatg | tcttgagtga | ggaaaacctt | tctagcaccc |
| | tgtgcctagg | | | | |
| 241 | cctcttccaa | ataacactgg | ctctcatcct | gggaaaacag | aggacc |
| // | | | | | |

| | |
|---|---|
| LOCUS | Y081818S07 361 bp DNA linear PRI |
| 04-APR-2002 | |
| DEFINITION | Homo sapiens suppressor of fused (SUFU) gene, exon 7. |
| ACCESSION | AY081824 |
| VERSION | AY081824 |
| KEYWORDS | . |
| SEGMENT | 7 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 361) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | signaling |
| JOURNAL | Unpublished |
| REFERENCE | 2 (bases 1 to 361) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | Sick Children, 88 Elm Street, Toronto, Ont M5G 1X8, Canada |
| FEATURES | Location/Qualifiers |
| source | 1..361 |
| | /organism=“Homo sapiens” |
| | /db_xref=“taxon:9606” |
| | /chromosome=“10” |
| | /map=“10q24” |
| exon | 123..276 EXON 7 |
| | /gene=“SUFU” |
| | /number=7 |
| BASE COUNT | 77 a 104 c 100 g 80 t |
| ORIGIN | |

| | | | | | |
|---|---|---|---|---|---|
| 1 | ttgtcccatg | ctcagcacca | caagggctca | gtaaatactg | taagagcagt |
| | ggctgaaagg | | | | |
| 61 | gtggtcacct | tgggtcacca | gttctctgaa | agaactctgg | ctctttggtt |
| | cttttcaagc | | | | |
| 121 | aggagagagt | tgacaaaggc | atcgagacag | atggctccaa | cctgagtggt |
| | gtcagtgcca | | | | |
| 181 | agtgtgcctg | ggatgacctg | agccggcccc | ccgaggatga | cgaggacagc |
| | cggagcatct | | | | |
| 241 | gcatcggcac | acagccccgg | cgactctctg | gcaaaggtgg | gagccatcac |
| | tcagcattcc | | | | |
| 301 | accagccttc | ctccttcctt | ttccccaggg | cctggtttcc | agtctctcta |
| | ggatgggtct | | | | |
| 361 | c | | | | |
| // | | | | | |

| | |
|---|---|
| LOCUS | Y081818S08 274 bp DNA linear PRI |
| 04-APR-2002 | |
| DEFINITION | Homo sapiens suppressor of fused (SUFU) gene, exon 8. |
| ACCESSION | AY081825 |
| VERSION | AY081825 |
| KEYWORDS | . |
| SEGMENT | 8 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 274) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 274) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..274 |
| | /organism=“Homo sapiens” |
| | /db_xref=“taxon:9606” |
| | /chromosome=“10” |
| | /map=“10q24” EXON 8 |
| exon | 73..184 |
| | /gene=“SUFU” |
| | /number=8 |
| BASE COUNT | 66 a 75 c 83 g 50 t |
| ORIGIN | |

| | | | | | |
|---|---|---|---|---|---|
| 1 | aggtttcaag | agcggggtga | gaattgctgg | gagcccactg | ggccactggg |
| | caacttagtg | | | | |
| 61 | gtgtcgttgc | agacacagag | cagatccggg | agaccctgag | gagaggactc |
| | gagatcaaca | | | | |
| 121 | gcaaacctgt | ccttccacca | atcaaccctc | agcggcagaa | tggcctcgcc |
| | cacgaccggg | | | | |
| 181 | ccccgtaagt | tccccagtgt | ccctgggctg | gaacaagagg | acgacttttt |
| | tctgaagggc | | | | |
| 241 | ctgtccctgt | ggattgcatg | agagagaaca | atgc | |
| // | | | | | |

| | |
|---|---|
| LOCUS | Y081818S09 266 bp DNA linear PRI |
| 04-APR-2002 | |
| DEFINITION | Homo sapiens suppressor of fused (SUFU) gene, exon 9. |
| ACCESSION | AY081826 |
| VERSION | AY081826 |
| KEYWORDS | . |
| SEGMENT | 9 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 266) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 266) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..266 |
| | /organism=“Homo sapiens” |
| | /db_xref=“taxon:9606” |
| | /chromosome=“10” |
| | /map=“10q24” |
| exon | 52..186 EXON 9 |
| | /gene=“SUFU” |
| | /number=9 |
| BASE COUNT | 58 a 93 c 61 g 54 t |
| ORIGIN | |

| | | | | | |
|---|---|---|---|---|---|
| 1 | tccctgagct | tttcaccttg | tgccgaacct | tttcctgtgc | ttgcttcaca |
| | ggagccgcaa | | | | |
| 61 | agacagcctg | gaaagtgaca | gctccacggc | catcattccc | catgagctga |
| | ttcgcacgcg | | | | |
| 121 | gcagcttgag | agcgtacatc | tgaaattcaa | ccaggagtcc | ggagccctca |
| | ttcctctctg | | | | |
| 181 | cctaaggtga | gcgagacagc | cctgccacac | agtttacccc | acagcaccca |
| | gctcagcctc | | | | |
| 241 | cagggggcac | ttcagagcct | ccccag | | |
| // | | | | | |

| | |
|---|---|
| LOCUS | Y081818S10 375 bp DNA linear PRI |
| 04-APR-2002 | |
| DEFINITION | Homo sapiens suppressor of fused (SUFU) gene, exon 10. |
| ACCESSION | AY081827 |
| VERSION | AY081827 |
| KEYWORDS | . |
| SEGMENT | 10 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 375) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 375) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..375 |
| | /organism=“Homo sapiens” |
| | /db_xref=“taxon:9606” |
| | /chromosome=“10” |
| | /map=“10q24” |
| exon | 65..203 |
| | /gene=“SUFU” EXON 10 |
| | /number=10 |
| BASE COUNT | 62 a 113 c 96 g 104 t |
| ORIGIN | |

| | | | | | |
|---|---|---|---|---|---|
| 1 | gcctgctgtg | cttggaactg | tttccaagcc | cagctcctca | ctgtctccat |
| | gttcccatct | | | | |
| 61 | ccaggggcag | gctcctgcat | ggacggcact | ttacatataa | aagtatcaca |
| | ggtgacatgg | | | | |
| 121 | ccatcacgtt | tgtctccacg | ggagtggaag | gcgcctttgc | cactgaggag |
| | catccttacg | | | | |
| 181 | cggctcatgg | accctggtta | caagtgagaa | ggcccttttt | cttctccctc |
| | cttcctttca | | | | |
| 241 | tagacttcct | tgcccacccc | tcctcttctc | ccttggcagc | tcttgatggc |
| | accccttcct | | | | |
| 301 | ggggggctgg | tcatgaatgc | ctcatggatt | cagggcctgg | ggcctgtgtg |
| | taggtatgga | | | | |
| 361 | gtgtggatgc | tgcta | | | |
| // | | | | | |

| | |
|---|---|
| LOCUS | Y081818S11 275 bp DNA linear PRI |
| 04-APR-2002 | |
| DEFINITION | Homo sapiens suppressor of fused (SUFU) gene, exon 11. |
| ACCESSION | AY081828 |
| VERSION | AY081828 |
| KEYWORDS | . |
| SEGMENT | 11 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 275) |
| AUTHORS | Taylor, M. D., Hogg, D. and Rutka, J. T. |
| TITLE | Mutations of Human Suppressor of Fused Predispose to |
| | Medulloblastoma Through Alterations in Sonic Hedgehog and Wnt |
| | Signaling |
| JOURNAL | Unpublished |
| REFERENCE | 2 (bases 1 to 275) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..275 |
| | /organism=“Homo sapiens” |
| | /db_xref=taxon:9606” |
| | /chromosome=“10” |
| | /map=“10q24” |
| exon | 132..206 |
| | /gene=“SUFU” |
| | /number=11 |
| BASE COUNT | 73 a 56 c 66 g 80 t |
| ORIGIN | EXON 11 |

| | | | | | |
|---|---|---|---|---|---|
| 1 | cttccctgtg | tcccttgaac | agatcacagt | gagctcatct | ctcctccatg |
| | gtcagaagag | | | | |
| 61 | aggtataacg | cttggtggtt | ggcaaaaaga | tcatacattt | aaaaataata |
| | ataaaagcct | | | | |
| 121 | gccttgtgcc | ttcacagatt | ctgttgaccg | aagagtttgt | agagaaaatg |
| | ttggaggatt | | | | |
| 181 | tagaagattt | gacttctcca | gaggaagtaa | gcttgtttga | cttttcctga |
| | caacaggtcc | | | | |
| 241 | cgtctctggg | accatgtgtg | tgcgtgcgtg | tgcac | |
| // | | | | | |

| | |
|---|---|
| LOCUS | Y081818S12 280 bp DNA linear PRI |
| 04-APR-2002 | |
| DEFINITION | Homo sapiens suppressor of fused (SUFU) gene, exon 12 and |
| | complete |
| | cds. |
| ACCESSION | AY081829 |
| VERSION | AY081829 |
| KEYWORDS | . |
| SEGMENT | 12 of 12 |
| SOURCE | human. |
| ORGANISM | Homo sapiens |
| | Eukaryota; Metazoa; Chordata; Craniata; Vertebrata; |
| | Euteleostomi; |
| | Mammalia; Eutheria; Primates; Catarrhini; Hominidae; Homo. |
| REFERENCE | 1 (bases 1 to 280) |
| AUTHORS | Taylor, M. D., Hogg, D. and Rutka, J. T. |
| TITLE | Mutations of Human Suppressor of Fused Predispose to |
| | Medulloblastoma through Alterations in Sonic Hedgehog and Wnt |
| | Signaling |
| JOURNAL | Unpublisbed |
| REFERENCE | 2 (bases 1 to 280) |
| AUTHORS | Taylor, M. D., Hogg, D. and Rutka, J. T. |
| TITLE | Direct Submission |
| JOURNAL | Submitted (04-MAR-2002) Brain Tumour Research Center, Hospital |
| | for |
| | Sick Children, 88 Elm Street, Toronto, Ont M5G 1X8, Canada |
| FEATURES | Location/Qualifiers |
| source | 1..280 |
| | /organism=“Homo sapiens” |
| | /db_xref=“taxon:9606” |
| | /chromosome=“10” |
| | /map=“10q24” |
| mRNA | |

join (AY081818:<305..486,AY081819:77..211,AY081820:79..215,

| | |
|---|---|
| | AY081821:95..237,AY081822:49..134,AY081823:123..135, |
| | AY081824:123..276,AY081825:73..184,AY081826:52..136, |
| | AY081827:65..203,AY081828:138..206,112..>201) |
| | /gene=“SUFU” |
| | /product=“suppressor of fused” |
| gene | |

order(AY081818:<305..623,AY081819:1..264,AY081820:1..275,

| | |
|---|---|
| | AY081821:1..400,AY081822:1..205,AY081823:1..286, |
| | AY081824:1..361,AY081825:1..274,AY081826:1..266, |
| | AY081827:1..375,AY081828:1..275,1..>201) |
| | /gene=“SUFU” |
| CDS | |

join(AY081818:305..456,AY081819:77..211,AY081820;79..215,

AY081821:95..237,AY081822:49..134,AY081823:123..195,
AY081824:123..276,AY081825:73..184,AY081826:52..186,
AY081827:65..203,AY081828:138..206,112..201)
/gene=“SUFU”
/codon_start=1
/product=“suppressor of fused”
/protein_id=“AAM08947”

/translation=“MAELRPSGAPGPTAPPAPGPTAPFAFASLFPPGLHAIYGECRRL
YPDQPNPLQVTAIVKYWLGGPDPLDYVSMYRNVGSPSANIPEHWHYISFGLSDLYGDN
RVHEFTGTDGFSGFGFELTFRLKRETGESAPPTWPAELMQGLARYVFQSENTFCSGDH
VSWHSPLDNSESRIQHMLLTEDPQMQPVQTPFGVVTFLQIVGVCTEELHSAQQWNGQG
ILELLRTVPIAGGPWLITDMRRGETIFEIDPHLQERVDKGIETDGSNLSGVSAKCAWD
DLSRPPEDDEDSRSICIGTQPRRLSGKDTEQIRETLRRGLEINSKPVLPPINPQRQNG
LAHDRAPSRKDSLESDSSTAIIPHELIRTRQLESVHLKFNQESGALIPLCLRGRLLHG
RHFTYKSITGDMAITFVSTGVEGAFATEEHPYAAHGPWLQILLTEEFVEKMLEDLEDL

| | |
|---|---|
| | TSPEEFKLPKEYSWPEKKLKVSILPDVVFDSPLH” |
| exon | 112..>201 |
| | /gene=“SUFU” |
| | /number=12 |
| BASE COUNT | 57 a 90 c 72 g 61 t |
| ORIGIN | |

| | | | | | |
|---|---|---|---|---|---|
| 1 | ggcctgtcct | atccctagct | ccccggggac | aggcctgggc | aatctctgga |
| | aagaccacgg | | | | |
| 61 | tgtattctgc | taaccactca | cactcctggt | ctgtgcttgc | tccctccaca |
| | gttcaaactt | | | | |
| 121 | cccaaagagt | acagctggcc | tgaaaagaag | ctgaaggtct | ccatcctgcc |
| | tgacgtggtg | | | | |
| 181 | ttcgacagtc | cgctacacta | gcctgggctg | ggccctgcag | tggccagcag |
| | ggagcccagc | | | | |
| 241 | tgctccccag | tgacttccag | tgtaacagtt | gtgtcaacga | |
| // | | | | | |

1. A method for determining a diagnosis, prognosis or risk of a tumor pathology in a subject involving a SUFU gene mutation, said method comprising detecting a SUFU gene mutation in DNA from said subject, wherein said SUFU gene comprises exons 1 through 12 and said mutation is associated with said tumor pathology.

2. The method of claim 1, wherein said mutation is present in exon 8 or 9 of said SUFU gene.

3. The method of claim 1, wherein said mutation is present in exon 1 or 2 of said SUFU gene.

4. The method of claim 1, wherein said mutation is a deletion of a portion of said SUFU gene.

5. The method of claim 4, wherein said deletion is a deletion of seven nucleotides from exon 9.

6. The method of claim 4, wherein said deletion is a N-carboxy terminal deletion.

7. The method of claim 1, wherein said mutation is selected from the group consisting of:
IVS8+1G→A, IVS1-1G→T, 1129delTCCGGAG, IVS1-1A→T, E1 143insA, a 2.5-2.8 Mb deletion on chromosome 10q and SUFU(212-484) being a N-terminal deletion, and combinations thereof.

8. The method of claim 1, wherein said mutation is a missense mutation selected from C44T and G1018T.

9. The method of claim 1, wherein said DNA is genomic DNA or cDNA.

10. The method of claim 9, wherein a mutation is detected by an assay selected from the group consisting of probe hybridization, direct sequencing, restriction enzyme fragment analysis and fragment electrophoretic mobility.

11. The method of claim 1, wherein said tumor pathology is selected from the group consisting of medulloblastoma, nevoid basal cell carcinoma, basal cell carcinoma, meningioma, colon cancer, muscle tumors and sarcomas.

12. The method of claim 1, wherein said method further comprises detecting expression of a SUFU gene product from said genomic DNA by a method selected from the group consisting of electrophoresis, HPLC, TLC, immunodiffusion, immunoelectrophoresis, RIA, ELISA, immunofluorescence and western blotting.

13. A method for determining whether a subject is at risk for development of medulloblastoma, the method comprising the steps of:
(a) obtaining a nucleic acid sample from the subject; and
(b) conducting an assay on the nucleic acid sample to determine the presence or absence of a Suppressor-of-Fused (SUFU) gene mutation associated with medulloblastoma, wherein the presence of a SUFU gene mutation associated with medulloblastoma indicates that the subject is at risk for development of medulloblastoma.

14. The method of claim 13, wherein the assay is selected from the group consisting of probe hybridization, direct sequencing, restriction enzyme fragment analysis and fragment electrophoretic mobility.

15. The method of claim 14, wherein the nucleic acid sample is an RNA sample and the assay is a direct sequencing assay.

16. The method of claim 14, wherein the assay comprises the steps of:
(a) reverse transcribing the RNA sample to produce a corresponding cDNA;
(b) performing at least one polymerase chain reaction with suitable oligonucleotide primers to amplify the SUFU cDNA;
(c) obtaining the nucleotide sequence of the amplified SUFU cDNA; and
(d) determining the presence or absence of a SUFU gene mutation associated with NBCCS in said nucleotide sequence.

17. The method of claim 16, wherein step (d) comprises determining the presence or absence of a mutation of the polynucleotide of Table 2, the mutation being selected from the group consisting of: IVS8+1G→A, IVS1-1G→T, 1129delTCCGGAG, IVS1-1A→T, E1 143insA, a 2.5-2.8 Mb deletion on chromosome 10q and SUFU(212-484) being a N-terminal deletion, and combinations thereof.

18. The method of claim 14, wherein the nucleic acid sample is a DNA sample.

19. The method of claim 18, wherein the DNA sample is a genomic DNA sample and the assay comprises the steps of:
(a) amplifying a target portion of the nucleotide sequence of the genomic DNA;
(b) obtaining the nucleotide sequence of said amplified target portion; and
(c) determining the presence or absence of a SUFU gene mutation associated with medulloblastoma in said target portion nucleotide sequence.

20. The method of claim 19, wherein step (c) comprises determining the presence or absence of a mutation of the polynucleotide of the SUFU nucleic acid sequence shown in Table 2, the mutation being selected from the group consisting of: IVS8+1G→A, IVS1-1G→T, 1129delTCCGGAG, IVS1-1A→T, E1 143insA, a 2.5-2.8 Mb deletion on chromosome 10q and SUFU(212-484) being a N-terminal deletion, and combinations thereof.

21. A method for determining whether a subject displaying a medulloblastoma phenotype, the method comprising the steps of:
(c) obtaining a nucleic acid sample from the subject;
(d) conducting an assay on the nucleic acid sample to determine the presence or absence of a SUFU gene mutation associated with medulloblastoma, wherein the presence of a SUFU gene mutation associated with medulloblastoma indicates that the subject is suffering from medulloblastoma.

22. The method of claim 21, wherein the assay is selected from the group consisting of probe hybridization, direct sequencing, restriction enzyme fragment analysis and fragment electrophoretic mobility.

23. The method of claim 22, wherein the nucleic acid sample is an RNA sample and the assay is a direct sequencing assay.

24. The method of claim 23, wherein the assay comprises the steps of:
(a) reverse transcribing the RNA sample to produce a corresponding cDNA;
(b) performing at least one polymerase chain reaction with suitable oligonucleotide primers to amplify the SUFU cDNA;
(c) obtaining the nucleotide sequence of the amplified SUFU cDNA;
and
(d) determining the presence or absence of a SUFU gene mutation associated with NBCCS in said nucleotide sequence.

25. The method of claim 24, wherein step (d) comprises determining the presence or absence of a mutation of the polynucleotide shown in Table 2, the mutation being selected from the group consisting of: IVS8+1 G→A, IVS1-1G→T, 1129delTCCGGAG, IVS1-1A→T, E1 143insA, a 2.5-2.8 Mb deletion on chromosome 10q and SUFU(212-484) being a N-terminal deletion, and combinations thereof.

26. The method of claim 25, wherein the nucleic acid sample is a DNA sample.

27. The method of claim 26, wherein the DNA sample is a genomic DNA sample and the assay comprises the steps of:
(a) amplifying a target portion of the nucleotide sequence of the genomic DNA;
(b) obtaining the nucleotide sequence of said amplified target portion; and
(c) determining the presence or absence of a SUFU gene mutation associated with NBCCS in said target portion nucleotide sequence.

28. The method of claim 27, wherein step (c) comprises determining the presence or absence of a mutation of the polynucleotide shown in Table 2, the mutation being selected from the group consisting of: IVS8+1 G→A, IVS1-1G→T, 1129delTCCGGAG, IVS1-1A→T, E1 143insA, a 2.5-2.8 Mb deletion on chromosome 10q and SUFU(212-484) being a N-terminal deletion, and combinations thereof.

29. The method of claim 1, wherein the subject is a human.

30. A method for treating a subject bearing a mutated SUFU gene, comprising administering to the subject an effective amount of an agent selected from the group consisting of:
(a) a nucleotide sequence encoding a normal SUFU gene;
(b) normal SUFU protein or an effective fragment thereof;
(c) a compound which inhibits SHH signalling; and
(d) an antibody that binds to a mutant SUFU protein.

31. The method of claim 30, wherein a SUFU protein encoded by the nucleic acid SUFU sequence of Table 2 is administered to the subject.

32. The method of claim 30, wherein the subject is a human.

33. A method for screening a candidate compound for its potential as a therapeutic for improvement of SUFU function in a subject having a mutated SUFU gene, comprising screening the candidate compound for its ability to inhibit SHH signalling, wherein an ability to inhibit SHH signalling indicates that the compound is a potential therapeutic for said subject.

34. An isolated human SUFU gene comprising 12 exons, said gene having a mutation in one or more of exons 1, 2, 8 or 9, wherein said mutation is indicative of a medulloblastoma phenotype.

35. An isolated human SUFU gene comprising one or more mutations selected from the group consisting of: IVS8+1GA, IVS1-1G→T, 1129delTCCGGAG, IVS1-1A→T, E1 143insA, a 2.5-2.8 Mb deletion on chromosome 10q and SUFU(212-484) being a N-terminal deletion, and combinations thereof.

36. A kit for the detection and/or quantification of SUFU gene or gene product by the method of claim 16, the kit comprising one or more nucleic acids and amplification primers and instructions for the use.
